(19) United States
(12) Patent Application Publication
(10) Pub. No.: US 2015/0352373 A1
(43) Pub. Date: Dec. 10, 2015

(54) AN APPARATUS TO DELIVER CONFORMAL RADIOTHERAPY USING EXTERNAL BEAM COBALT 60

(71) Applicant: PANACEA MEDICAL TECHNOLOGIES PVT. LTD, Bangalore, Karnataka (IN)

(72) Inventors: Goteti Vankata Subrahmanyam, Bangalore, Karnataka (IN); Deepak Fernando, Bangalore, Karnataka (IN); Tamilarasan Mani, Bangalore, Karnataka (IN); Kuppa Avinash Rao, Bangalore, Karnataka (IN)

(21) Appl. No.: 14/761,889

(22) PCT Filed: Jan. 16, 2014
(86) PCT No.: PCT/IB14/58313

§ 371 Date: Jul. 17, 2015

(30) Foreign Application Priority Data
Jan. 17, 2013 (IN) 230/CHE/2013

(51) Int. Cl. A61N 5/10

(52) U.S. Cl. CPC A61N 5/1045; A61N 5/1081; A61N 5/1049; A61N 2005/1094; A61N 2005/1061; A61N 2005/1091

(57) ABSTRACT

An apparatus to deliver conformal radiotherapy using external beam Cobalt 60 source is disclosed. The apparatus comprises an enclosed circular gantry placed above the bearings along with an integrated in house X-ray unit with the X-ray detector, source head and beam stopper. The X-ray unit along with the X-ray detector of the enclosed circular gantry are aligned to the radiation beam plane such that 3-D image of the tumour can be constructed using a cone beam computed tomography (CBCT) and radiation treatment is delivered by beam limiting device, without moving the patient from one patients treatment couch to other thus reducing the inconvenience to the patient achieving high tumour positioning accuracy and for alignment of X-ray and radiation beams.

### TECHNICAL FIELD OF THE INVENTION

The present invention relates to an apparatus that delivers conformal radiotherapy using external beam Cobalt 60. In particular, the present invention relates to radiotherapy apparatus which provides high tumour positioning accuracy and reduces the need for frequent calibration for alignment of X-ray and radiation beams.

### BACKGROUND OF THE INVENTION

Radiation therapy has seen several advancements in the past decade. Many of these have been developed by improvements in computers, imaging, dose computations, treatment planning tools, and beam-shaping hardware. Intensity-modulated radiation therapy (IMRT) was the next logical step, which provided the physicist the ability to conform the dose distributions to treat desired targets while sparing normal structures. Co-60 has been used extensively in the past for traditional, non-IMRT radiation therapy.

Radiation therapy itself can be classified according to two primary types, internal and external radiation therapy. External beam radiotherapy or teletherapy is the most common form of radiotherapy where the patient sits or lies on a couch and an external source of radiation is administrated through a machine which is capable of producing high energy external beam radiation. This therapy can include either total body irradiation or can be localized to the region of the tumor. The radiation itself can be either electromagnetic (X-ray or gamma radiation) or particulate (α or β particles). Internal radiation therapy, also termed brachytherapy, involves implantation of a radioactive isotope as the source of the radiation. There are a variety of methods of delivering internal radiation sources, including but not limited to, permanent, temporary, sealed, unsealed, intracavity or interstitial implants. The choice of implant is determined by a variety of factors including the location and extent of the tumor. Internally delivered radiation includes therapeutically effective radioisotopes injected into a patient. Such radioisotopes include, but are not limited to, radionuclide metals such as ¹⁸⁶RE, ¹⁸⁸RE, ⁶⁴Cu, ⁹⁰ytrium, ¹⁰⁹Pd, ²¹²Bi, ²⁰³Pb, ²¹²Pb, ²⁰³Pb, ²¹²Pb, ²¹¹At, ⁹⁷Ru, ¹⁰⁵Rh, ¹⁹⁸Au, ¹⁹⁹Ag and ¹³¹I. These radioisotopes generally will be bound to carrier molecules when administered to a patient.

Currently linear accelerators (linacs) medical units are used in external beam radiation therapy to treat cancer. Linacs emit a well defined beam of X-ray photon radiation of different energies or electron beams radiation depending on the accelerator used in the treatment. The medical units consist of four major components; a modulator, an electron gun, a Radio Frequency (RF) power source, and an accelerator guide. The electron beam produced by a linac can be used for treatment or can be directed toward a metallic target to produce X-rays. The modulator amplifies the AC power supply, rectifies it to DC power, and produces high-voltage DC pulses that are used to power the electron gun and RF power source. High-voltage cables electrically connect the electron gun and RF power source to the modulator, which can be located in the gantry, the gantry supporting stand, or a separate cabinet.

Intensity-modulated radiation therapy (IMRT) is an advanced mode of high-precision radiation therapy that utilizes computer controlled X-ray accelerators to deliver precise radiation doses to malignant tumors. The radiation dose is designed to conform to the three-dimensional (3-D) shape of a tumor by modulating the intensity of the radiation beam to focus a higher radiation dose to the tumor while minimizing radiation exposure to surrounding normal tissue. One form of IMRT is known as tomotherapy therapy wherein tomotherapy achieves dose conformity to a tumor by modulating the intensity of a fan beam of radiation as the source revolves about a patient. The intensity of radiation is modulated using a multi-leaf collimator, which is made up of individual leaves of a high atomic number material, usually tungsten that can move in and out of the field to produce a sequence of complex field shapes or beam apertures. Currently available tomotherapy machines use X-ray linear accelerators (linacs) as a source of radiation. However, since linacs are technologically complex and expensive the world wide use of linac based tomotherapy is limited.

US Publication No. US2002006182A1 discloses a technique for Intensity Modulated Radiation Therapy (IMRT) in an advanced form of external beam irradiation that is commonly referred as three-dimensional conformal radiation therapy (3DCRT), uses an array of radiation sources disposed within cells of a two-dimensional (2D) grid of radiation blocking walls. The intensity of radiation applied to a patient from any individual source is modulated dependent on local properties of a target, such as characteristics or dimensions of that portion of a tumor subject to radiation from a given source at a given time. Also arrangements of the major components in the invention are same as in medical linear accelerator.

Multileaf collimator (MLC) has been the main tool for radiation therapy dose delivery and is now widely used for Intensity modulated radiation therapy. It is a simple and useful system in the preparation and performance of radiotherapy treatment. Multileaf collimators are reliable, as their manufacturers developed various mechanisms for their precision, control and reliability, together with reduction of leakage and transmission of radiation between and through the leaves. Multileaf collimator is known today as a very useful clinical system for simple field shaping, but its use is getting even more important in dynamic radiotherapy, with the leaves moving during irradiation. But currently used MLC's in radiotherapy devices can deliver either a field shaped dose delivery or an intensity modulated beam.

Radiation therapy can be either curative or palliative, depending on the stage and prognosis of the disease. For successful treatment, the radiation field must be very carefully delivered and well defined to avoid irradiating healthy tissue .In contrast to internal radiotherapy in which the radiation source is inside the body, external beam radiotherapy directs the radiation at the tumor from outside the body.

Cobalt-60 machines are more suitable for the radiotherapy, considering the cost and maintenance issues. As the radiation is harmful to healthy cells as well, it is required to have a mechanism which can deliver specific amounts of radiation only to the designated area of the body, while keeping the exposure to the adjacent areas limited to the extent possible. Concern for radiation protection and regulations is growing significantly because of rapid increase in use of radiation and better understanding of the risks and benefits attributable to it.

Hence, what is needed is a reliable device that delivers conformal radiotherapy using external beam Cobalt 60 and that is simple to maintain compared to the modern linear accelerator.

### SUMMARY OF THE INVENTION

The present invention overcomes the drawback of prior art by providing an apparatus that delivers conformal radiotherapy using external beam Cobalt 60 that is simple to maintain compared to the modern linear accelerator. The apparatus of the present invention also provides intensity modulated radiotherapy (IMRT) based on the dynamic use of MLC.

The present invention relates to an apparatus that delivers conformal radiotherapy using external beam Cobalt 60 as a source. Furthermore, the apparatus of the present invention uses Image Guided Radiotherapy Treatment (Conformal Gamma therapy) Planning System (IGRT-TPS) process for 3-D imaging during a course of radiation treatment and to direct radiation therapy utilizing the imaging coordinates of the actual radiation treatment plan. In present invention, IGRT-TPS is used along with cone beam CT to obtain 3-D positioning of the patient and tumor site. IGRT process is used to improve the accuracy of the radiation field placement and also to reduce the exposure of healthy tissue during radiation treatments.

The present invention comprises a base support structure with gantry support bearings. An enclosed circular gantry is placed above the bearings along with an integrated in house X-ray unit with the X-ray detector, source head and beam stopper. The X-ray unit along with the X-ray detector of the enclosed circular gantry are aligned to the radiation beam plane such that 3-D image of the tumour may be constructed using a cone beam computed tomography (CBCT). Furthermore, integrated X-ray system and cone beam CT based image guided system help in an online planning made by the physicist and radiation treatment is delivered, without moving the patient from one patients treatment couch to other thus reducing the inconvenience to the patient. The planning also achieves high tumour positioning accuracy and for alignment of X-ray and radiation beams.

In accordance with one embodiment of the present invention, cobalt radiotherapy circular gantry is provided with the central opening for passage of patients treatment couch. The patient lies on the patient treatment couch wherein imaging and radiation treatment is performed. Furthermore, the patient's treatment couch is provided with an additional movement such as pitch, yaw and roll which gives more flexibility in planning the treatment and also increases the treatment accuracy. Also, in the present invention the patient treatment couch enables the tumor to be at iso-center.

Another embodiment of the present invention discloses a beam limiting devices (BLD) mounted on the enclosed circular gantry. The beam limiting devices is used for dose delivery radiotherapy or Intensity Modulated Radiation Therapy (IMRT) which is an advanced and most precise form of external beam radiation therapy. BLD is constructed by using two banks of leaves mounted in the beam path along with linear motion, which are capable of delivering field shaped dose delivery and intensity modulated dose delivery. Hence the present invention is a multipurpose apparatus which delivers a pencil beam and a large beam combining two types of collimators in a single unit.

### BRIEF DESCRIPTION OF THE DRAWINGS

The foregoing and other features of embodiments will become more apparent from the following detailed description of embodiments when read in conjunction with the accompanying drawing. In a drawing, like reference numerals refer to like elements.

FIG. 1 illustrates the perspective view of an apparatus that delivers conformal radiotherapy using external beam Cobalt 60 in accordance with one embodiment of the present invention.

FIG. 2 illustrates the side view of an apparatus that delivers conformal radiotherapy using external beam Cobalt 60 in accordance with one embodiment of the present invention.

FIG. 3 represents the BLD (beam limiting device) mounted on an enclosed circular gantry in accordance to one embodiment of the present invention.

### DETAILED DESCRIPTION OF THE INVENTION

Reference will now be made in detail to the description of the present subject matter, one or more examples of which are shown in figures. Each example is provided to explain the subject matter and not a limitation. Various changes and modifications obvious to one skilled in the art to which the invention pertains are deemed to be within the spirit, scope and contemplation of the invention.

The term ‘conformal radiotherapy’ used herein refers to delivering a high radiation dose to a volume that closely conforms to the shape of the patient's tumor volume accurately. Conformal radiotherapy refers to accurately identifying both the exact shape and location of the tumor so as to distribute the radiation dose as close as possible to the margin around the target.

The present invention relates to an apparatus that delivers conformal radiotherapy using external beam Cobalt 60. In particular, the present invention relates to radiotherapy apparatus which provides high tumour positioning accuracy and reduces the need for frequent calibration for alignment of X-ray and radiation beams.

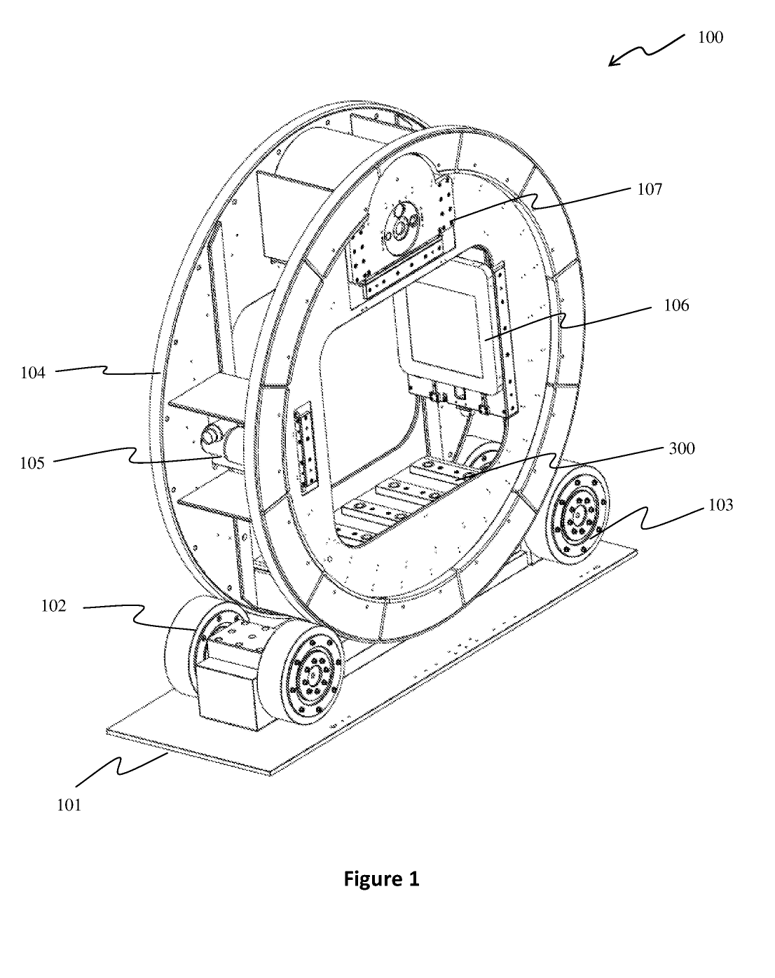

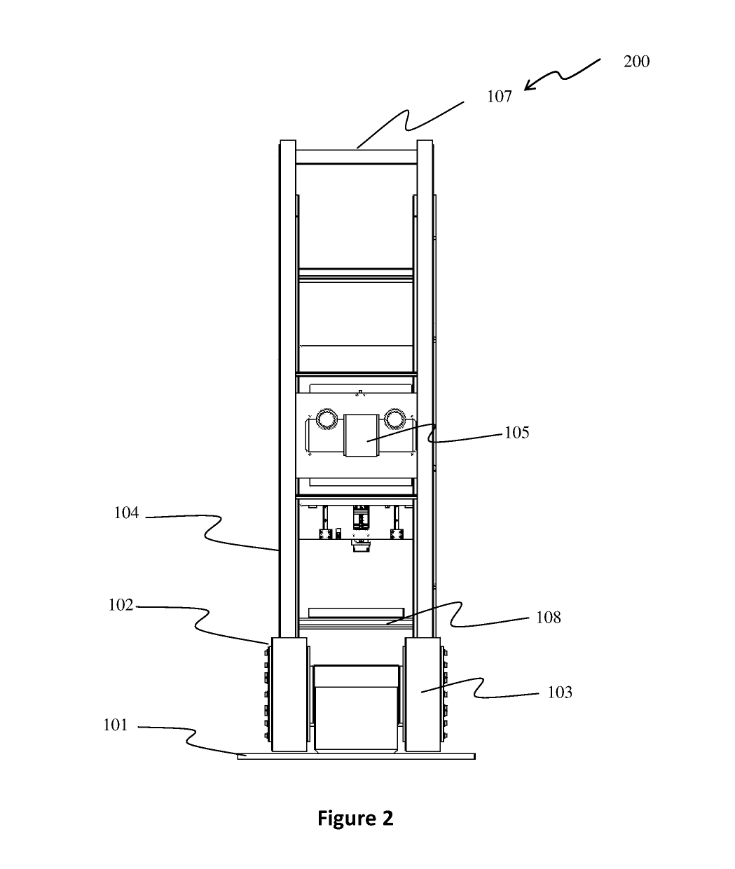

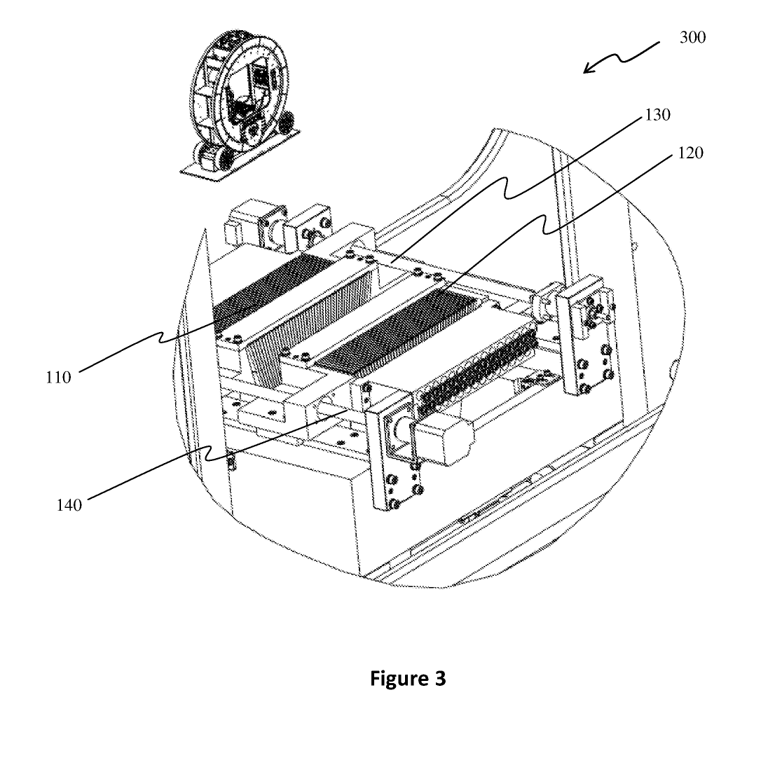

FIG. 1 illustrates the perspective view of an apparatus that delivers conformal radiotherapy using external beam Cobalt 60 in accordance to one embodiment of the invention. An apparatus that delivers conformal radiotherapy using external beam Cobalt 60 (100) comprises a base support structure (101) with gantry support bearings (102 and 103). Enclosed circular gantry (104) is placed above the bearings along with an integrated in house X-ray unit (105) with the X-ray detector (106), source head (107) and beam stopper (108). The enclosed circular gantry (104) of the present invention has a central passage adequate enough to allow the patients treatment couch with patient to move to-and-fro through the gantry longitudinally. The enclosed circular gantry (104) also houses in it, the imaging unit and radiotherapy unit. The X-ray unit (105) along with the X-ray detector (106) is housed in the enclosed circular gantry (104) and aligned normal to the radiation beam source head (107) for generating a 3-D image which ensures Image Guided Radiotherapy Treatment (Conformal Gamma therapy). A beam limiting device (BLD) (300, FIG. 3) is mounted on the enclosed circular gantry (104), wherein the beam limiting device (300, FIG. 3) has a plurality of bank of leafs (110 and 120, FIG. 3) to generate a field shaped dose delivery and intensity modulated beam for radiotherapy. The bearings (102 and 103) are used in the external beam cobalt 60 radiotherapy apparatus for supporting the enclosed circular gantry (104) and the bearings (102 and 103) also allows the operator to continuously rotate the enclosed circular gantry (104) along the treatment axis. The beam stopper (108, FIG. 2) absorbs the radiation coming out of the patient to reduce the shielding requirements of the treatment room and surrounding shielding areas that constitute as parts in and around the treatment facility. The source head (107) and beam stopper (108) are mounted on the enclosed circular gantry (104) and the source head (107) aligns with the X-ray beam. The X-ray detector (106) is mounted in the path of X-ray beam plane, on enclosed circular gantry (104) to construct a cone beam CT image.

FIG. 2 illustrates the side view of an apparatus that delivers conformal radiotherapy using external beam Cobalt 60 in accordance with one embodiment of the present invention. The beam stopper (108) is mounted on the enclosed circular gantry (104). The beam stopper (108) absorbs the radiation coming out of the patient to reduce the shielding requirements of the treatment room and the surrounding shielding areas that constitute as parts in and around the treatment facility.

FIG. 3 represents the beam limiting device mounted on an enclosed circular gantry in accordance to one embodiment of the present invention. The Beam Limiting Device (BLD) comprises a plurality of bank of leafs (110 and 120) mounted on an linear motion guide, wherein the linear motion guide further comprises a plurality of bank of leafs driven by individual motors to generate field based dose delivery and intensity modulated dose delivery. The plurality of bank of leafs (110 and 120) are driven by high speed motors, are moved to the centre of the beam.

It is to be understood, however, that even though numerous characteristics and advantages of the present invention have been set forth in the foregoing description, together with details of the structure and function of the invention, the disclosure is illustrative only. Changes may be made in the details, especially in matters of shape, size, and arrangement of parts within the principles of the invention to the full extent indicated by the broad general meaning of the terms in which the appended claims are expressed.

I claim:

1. An apparatus to deliver conformal radiotherapy using external beam cobalt 60 comprise of:
a) an enclosed circular gantry (104) comprising:
i. an in house X-ray unit (105) integrated with the enclosed circular gantry (104);
ii. the X-ray unit (105) has a X-ray detector (106), wherein the X-ray unit plane along with X-ray detector is aligned with a radiation beam plane to construct a 3-D image of the tumour using a cone beam CT technique;
iii. a beam limiting device (300) mounted on the enclosed circular gantry (104), wherein the beam limiting device (300) has a plurality of bank of leafs (110 and 120) to generate a field shaped dose delivery and intensity modulated beam for radiotherapy;
iv. a central opening in the enclosed circular gantry to facilitate to and fro movement of a patient's treatment couch through the enclosed circular gantry (104) longitudinally; and
v. a beam stopper (108) placed below a patient's treatment couch, wherein the beam stopper (108) is placed in the exact path of the radiation beam to absorb the radiation coming out of the patient, to reduce shielding requirements of a treatment room and surrounding shielding areas.

2. The apparatus as claimed in claim 1, wherein the enclosed circular gantry (104) is placed on a plurality of bearings (102 and 103) mounted on the base support structure (101).

3. The apparatus as claimed in claim 2, wherein the said bearings (102 and 103) firmly support the enclosed circular gantry (104) and facilitate an operator to continuously rotate the enclosed circular gantry along the axis of patients treatment couch during the process of treatment.

4. The apparatus as claimed in claim 1, wherein the beam limiting device (BLD) further comprises:
a. a plurality of linear motion guides (130 and 140); and
b. a plurality of bank of leafs (110 and 120) mounted on the linear motion guide, wherein the plurality of banks of leafs are driven by individual motors to generate field based on dose delivery and intensity modulated dose delivery for the treatment.

5. The apparatus as claimed in claim 4, wherein the bank of leafs (110 and 120) are moved on the linear motion guide (130 and 140) to the centre of the radiation beam to generate a field shaped dose delivery and intensity modulated beam.
